(19) United States
(12) Patent Application Publication
(10) Pub. No.: US 2017/0249871 A1
(43) Pub. Date: Aug. 31, 2017

(54) TRAINING CORNEA FOR REFRACTIVE SURGERY TRAINING

(71) Applicant: REVISION OPTICS, INC., Lake Forest, CA (US)

(72) Inventor: Alan Ngoc LE, Lake Forest, CA (US)

(21) Appl. No.: 15/508,499

(22) PCT Filed: Aug. 13, 2015
(86) PCT No.: PCT/US2015/044982

§ 371 Date: Mar. 3, 2017

Related U.S. Application Data
(60) Provisional application No. 62/048,666, filed on Sep. 10, 2014.

(51) Int. Cl. G09B 23/32

(52) U.S. Cl. CPC G09B 23/32; A61F 2/14

(57) ABSTRACT

A training cornea adapted to train physicians how to position a corneal inlay within a cornea, and methods thereof. The training cornea having a pre-made flap therein and having a base curvature with a radius of curvature that is the same as the radius of curvature training ball. The training cornea comprising a hydrophilic material.

### CROSS REFERENCE TO RELATED APPLICATIONS

This application claims priority to U.S. Prov. App. No. 62/048,666, filed Sep. 10, 2014, which is incorporated by reference herein.

This application is related to U.S. Pat. No. 8,057,541, issued Nov. 15, 2011; U.S. Pub. No. 2008/0262610, published Oct. 23, 2008; U.S. Pub. No. 2009/0198325, published Aug. 6, 2009; and U.S. Pub. No. 2011/0218623, published Sep. 8, 2011, the disclosures of which are incorporated by reference herein in their entireties. For example, the training devices and methods herein can be used for training physicians how to position any of the corneal inlays described in the references set forth above.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Physicians must train, or practice, positioning a corneal inlay within a cornea before they can position an inlay in a live patient. Traditionally, animal eyes are used as training eyes for the physicians. It would be beneficial to be able to train physicians how to position inlays within a cornea without having to use animal eyes.

SUMMARY OF THE DISCLOSURE

One aspect of the disclosure is a training cornea with a pre-made flap therein.

One aspect of the disclosure is a training cornea with a pre-made pocket therein.

The training cornea can be made from a hydrophilic material.

The training cornea can be at least 50% water, such as 78%.

The training cornea can have a pre-made flap, wherein the pre-made flap is 100 to 200 microns thick.

The training cornea can have a base curvature with a radius of curvature that is the same as the radius of curvature of a training ball.

The training cornea can have a front curvature with a radius of curvature that is 12.1 mm.

The training cornea can have a base curvature with a radius of curvature that is 12.5 mm.

The training cornea can have a diameter between 10 mm and 30 mm.

The training cornea can have a central thickness along the optical axis that is between 300 microns and 1000 microns.

The training cornea may not comprise animal corneal tissue.

One aspect of the disclosure is a packaging with a training cornea with a pre-made flap therein.

One aspect of the disclosure is a packaging with a training cornea with a pre-made pocket therein.

One method of the disclosure is a method of creating a flap in a training cornea, wherein the training cornea has not been placed into an eye of a patient. The method can further comprise placing the training cornea with the flap made therein into a packaging device.

One aspect of the disclosure is a method of creating a pocket in a training cornea, wherein the training cornea has not been placed into an eye of a patient. The method can further include placing the training cornea with the pocket made therein into a packaging device.

One aspect of the disclosure is a method of positioning a training cornea with a pre-made flap therein onto a training apparatus, and positioning an inlay under the pre-made flap.

One aspect of the disclosure is a method of positioning a training cornea with a pre-made pocket therein onto a training apparatus, and positioning an inlay into the pre-made pocket.

One aspect of the disclosure is an apparatus used in training ophthalmic procedures, comprising: a meniscus-shaped hydrogel body with a water content of at least 50%, the hydrogel body having a diameter between 10 mm and 30 mm, a center thickness between 300 and 1000 microns, a convex anterior surface and a concave posterior surface.

One aspect of the disclosure is a method of manufacturing a training cornea, comprising: machining a hydrogel body to have a meniscus shape, a diameter between 10 mm and 30 mm, a center thickness between 300 and 1000 microns, a convex anterior surface, and a concave posterior surface.

### BRIEF DESCRIPTION OF THE DRAWINGS

FIGS. 1A and 1B illustrate an exemplary training cornea after a flap has been made therein.

FIG. 2 is a side sectional view and illustrates an exemplary training cornea.

FIGS. 3A and 3B illustrate an exemplary training cornea with a pocket pre-made therein.

FIGS. 4A-4E illustrate an exemplary method of using a training cornea.

### DETAILED DESCRIPTION

Corneal inlays have been described that can be positioned under a corneal flap, or in a corneal pocket. There disclosure herein describes devices and methods for training medical personnel how to position corneal inlays within corneal tissue. The devices and methods herein can be used to train physicians how to position any of the corneal inlays described in, for example, U.S. Pat. No. 8,057,541, issued Nov. 15, 2011; U.S. Pub. No. 2008/0262610, published Oct. 23, 2008; U.S. Pub. No. 2009/0198325, published Aug. 6, 2009; and U.S. Pub. No. 2011/0218623, published Sep. 8, 2011, within a cornea. The devices and methods herein can be used to train physicians how to position other types of ocular devices in corneal tissue as well. The devices and methods herein are an alternative to using animal eyes to train physicians.

One aspect of the disclosure is a custom training cornea with a pre-made flap. In some embodiments the training cornea is made from a hydrophilic material that includes about 78% of fluid (e.g., water, Saline). In some embodiments the pre-made flap, measured from the anterior surface of the training cornea, is between 100 to 200 microns thick. The flap could have other thicknesses as well, such as between 5 and 50 microns, between 50 and 100 microns, between 200 and 250 microns, between 250 and 300 microns, between 300 and 350 microns, or between 350 and 400 microns, etc. The flap can be created with a femtosecond laser or mechanically.

As used herein, “training cornea” refers to a non-animal training cornea.

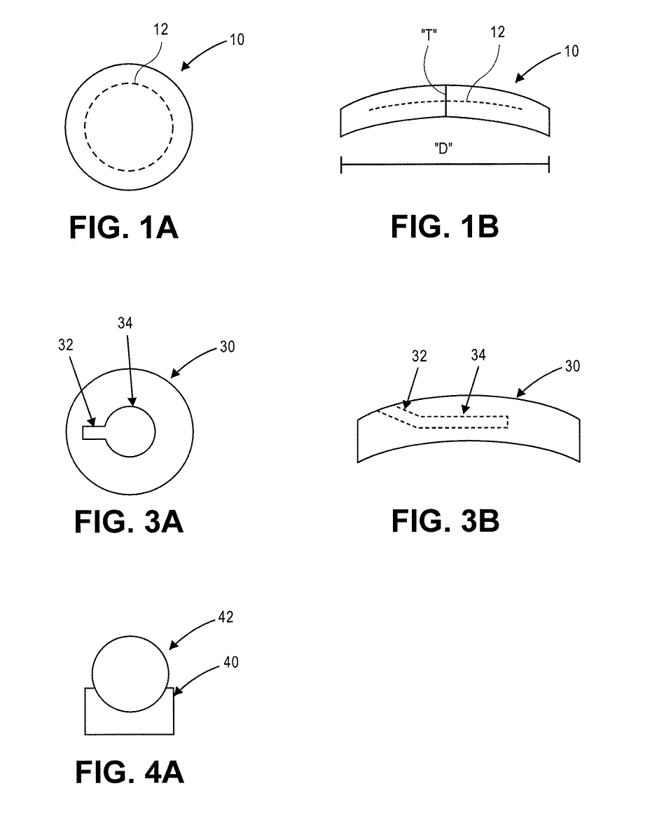

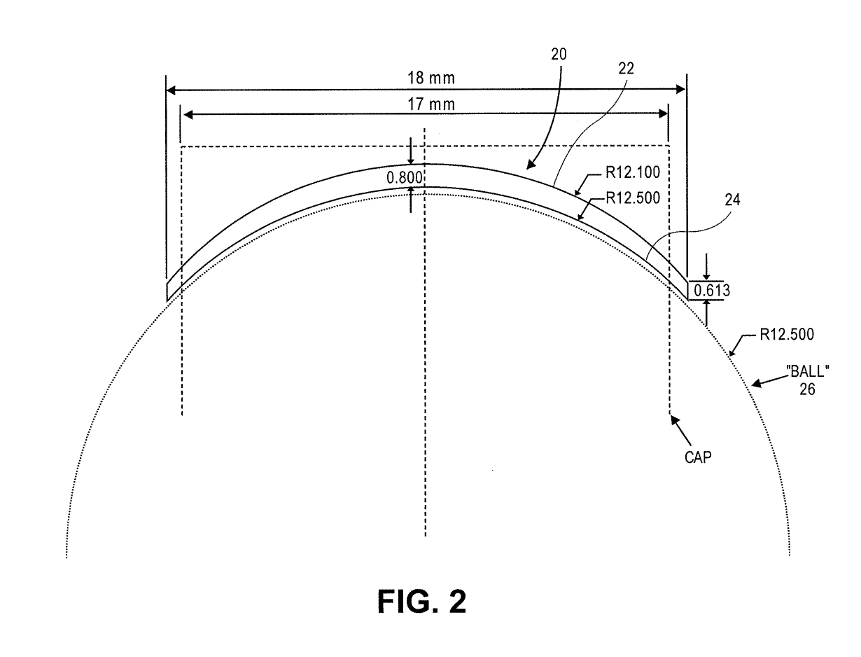

FIGS. 1A (top view) and 1B (sectional side view) illustrate an exemplary training cornea 10 after a flap 12 has been made therein. Flap 12 can be made with known techniques, such as with a femtosecond laser. In some embodiments the diameter (“D”) of training cornea is between 15 and 19 mm. In some embodiments the central thickness (“T”), measured along the axis of the training cornea, is between 300 microns and 1000 microns. FIG. 2 is a side sectional view and illustrates an exemplary training cornea 20. Training cornea 20 has a diameter of 18 mm, a central thickness of 0.8 mm, an edge thickness of 0.613 mm, base curve 24 radius of curvature of 12.5 mm, and a front curve 22 radius of curvature of 12.1 mm. Other aspects of FIG. 2 are described below.

In some embodiments the diameter is between 10 mm and 30 mm, such as, for example, between 10 mm and 25 mm, or between 10 mm and 20 mm, or between 12 mm and 25 mm, or between 15 mm and 30 mm, or between 15 mm and 25 mm.

In some embodiments the radii of curvature can be designed to be representative of an actual cornea, such as having the base radius of curvature be about 8 mm (e.g., 7.8 mm).

In some embodiments the training cornea is formed from a hydrogel material, polymerized using known techniques, and then lathed into the final configuration using known techniques. The training cornea generally has a high water content, such as at least 50% water. For example, in one specific embodiment it can be about 78% water.

One aspect of the disclosure is a custom training cornea with a pre-made pocket. In some embodiments the training cornea is made from a hydrophilic material that includes about 78% of fluid (e.g., water, Saline). In some embodiments the pocket includes an entrance dimension between 2 to 7 mm, and the pocket can be created by a femtosecond laser, techniques of which are known.

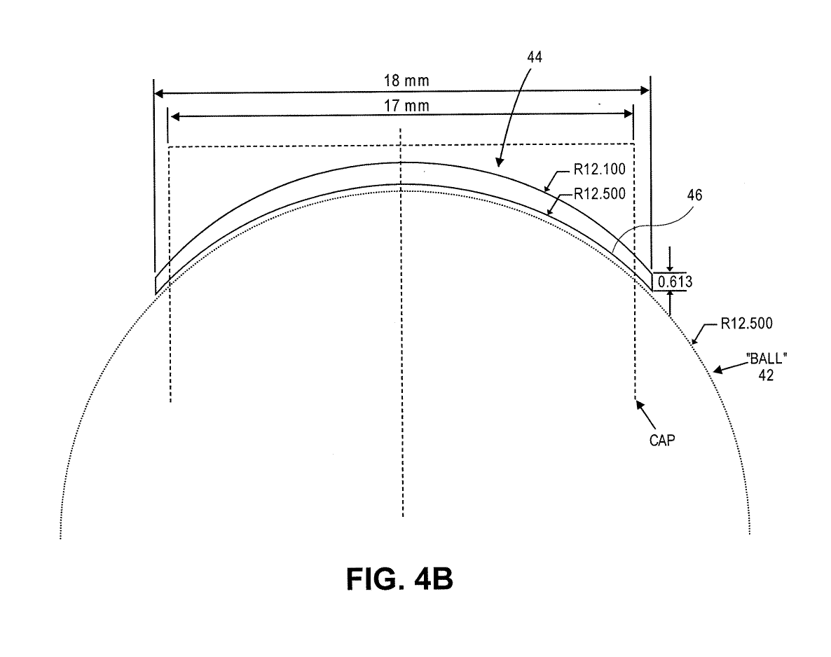

FIGS. 3A (top view) and 3B (sectional side view) illustrate an exemplary training cornea 30 with a pocket pre-made therein. The pocket includes entrance channel 32 and implant region 34.

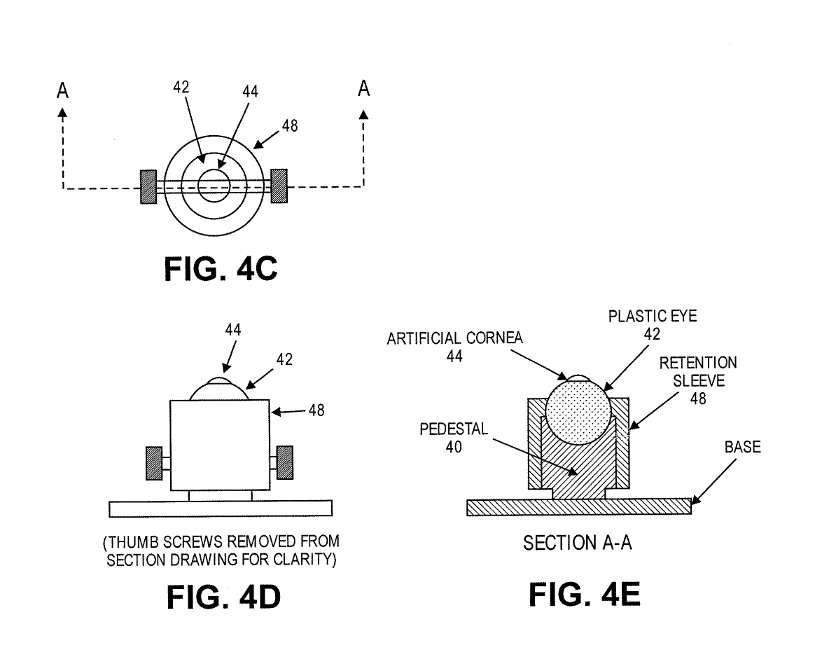

One aspect of the disclosure is a method of using a training cornea with a pre-made flap therein to train a physician to implant a corneal inlay under a corneal flap. An exemplary method is described in the context of FIGS. 4A-4C. FIG. 4A illustrates an exemplary first step in the method. Training ball 42 is placed onto fixture 40 (i.e., ball holder), and then the fixture and the ball are placed under the microscope. In this embodiment training ball 42 is a 1 inch polyurethane ball, although other types can be used.

As shown in the close up view of FIG. 4B, training cornea 44 is then positioned onto training ball 42 (fixture 40 not shown). In this embodiment the radius of curvature of ball 42 is the same as the radius of curvature of base curve 46 of the training cornea 44. In this embodiment they are both 12.5 mm. The training cornea can be stored in a separate packaging (e.g., bottle). The cap from the storage bottle is removed and tweezers can be used to transfer the training cornea onto the training ball into the position shown in FIG. 4B.

Next, as shown in FIGS. 4C-4E (FIG. 4C a top view, FIG. 4D a side view, and FIG. 4E being sectional view A-A from FIG. 4C), the cap or sleeve 48 for fixture 40 (also referred to as pedestal 40) is then placed onto the edge of training cornea 44 to secure the training cornea to the ball. With the training cornea secured to the ball using the cap, the training can begin. Under the microscope, the physician identifies the flap hinge on the training cornea. An instrument such as a spatula is used to separate the edge of the flap then retract the flap completely to the hinge. The method steps of positioning an inlay under the flap can vary, depending on the type of inlay. The steps of positioning the inlay onto the training corneal bed are not limiting and thus will not be described in detail. For example, an inlay can be positioned on the training cornea with forceps or tweezers. After the inlay is positioned onto the training corneal bed, the flap is placed back down over the inlay.

One aspect of the disclosure is a training method for positioning an inlay within a pre-made pocket in a training cornea to train a physician how to position an inlay within a corneal pocket. An exemplary training cornea with a pre-made pocket is shown in FIGS. 3A and 3B. The first steps are the same as if the training cornea has a pre-made flap, and thus FIGS. 4A and 4B and the descriptions thereof are referred to again.

Once under the microscope, the entrance of the pocket on the training cornea is identified. Then, with an instrument such as a spatula, the physician separates the edge of the entrance, then advances the spatula inside the pocket and separates the tissue to create the full pocket space. Optionally the pocket is hydrated to keep it hydrated. An inlay can then be positioned into the pocket using any known technique, and the disclosure is not limited to the manner in which the inlay is positioned in the pocket. For example, an inlay can be positioned into the pocket using any of the methods or devices described in U.S. Pat. No. 8,162,953, issued Apr. 24, 2012; U.S. Pub. No. 2013/0253527, published Sep. 26, 2013; and U.S. Provisional Application 61/980,504, filed Apr. 16, 2014, the disclosures of which are incorporated by reference herein.

The training corneas herein can also be used for calibrating the depth of cut from any femtosecond laser systems. For example, after a flap is made with a femtosecond laser and the flap lifted, the flap thickness can be measured using known techniques, and the laser system can then be calibrated based on the measured thickness.

As set forth here, the training corneas described herein are a potential substitute for any animal eyes (e.g., pig, cow, etc.) used for wet lab training in the ophthalmology field. Some kits are available but are designed for training of cataract surgery. In these kits the material is not designed for a femtosecond laser to make a flap or a pocket, and thus are not acceptable solutions.

1. A training cornea with a pre-made flap therein.

2. A training cornea with a pre-made pocket therein.

3. The training cornea of claim 1 wherein the training cornea is made from a hydrophilic material.

4. The training cornea of claim 1 wherein the training cornea is at least 50% water, such as 78%.

5. The training cornea of claim 1 wherein the pre-made flap is 100 to 200 microns thick.

6. The training cornea of claim 1 wherein the training cornea has a base curvature with a radius of curvature that is the same as the radius of curvature of a training ball.

7. The training cornea of claim 1 wherein the training cornea has a front curvature with a radius of curvature that is 12.1 mm.

8. The training cornea of claim 1 wherein the training cornea has a base curvature with a radius of curvature that is 12.5 mm.

9. The training cornea of claim 1 wherein the training cornea has a diameter between 10 mm and 30 mm.

10. The training cornea of claim 1 wherein the training cornea has a central thickness along the optical axis that is between 300 microns and 1000 microns.

11. The training cornea of claim 1 wherein the training cornea does not comprise animal corneal tissue.

12. A packaging with a training cornea with a pre-made flap therein.

13. A packaging with a training cornea with a pre-made pocket therein.

14. A method of creating a flap in a training cornea, wherein the training cornea has not been placed into an eye of a patient.

15. The method of claim 14 further comprising placing the training cornea with the flap made therein into a packaging device.

16. A method of creating a pocket in a training cornea, wherein the training cornea has not been placed into an eye of a patient.

17. The method of claim 16 further comprising placing the training cornea with the pocket made therein into a packaging device.

18. Positioning a training cornea with a pre-made flap therein onto a training apparatus, and positioning an inlay under the pre-made flap.

19. Positioning a training cornea with a pre-made pocket therein onto a training apparatus, and positioning an inlay into the pre-made pocket.

20. An apparatus used in training ophthalmic procedures, comprising:
a meniscus-shaped hydrogel body with a water content of at least 50%, the hydrogel body having a diameter between 10 mm and 30 mm, a center thickness between 300 and 1000 microns, a convex anterior surface and a concave posterior surface.

21. A method of manufacturing a training cornea, comprising:
machining a hydrogel body to have a meniscus shape, a diameter between 10 mm and 30 mm, a center thickness between 300 and 1000 microns, a convex anterior surface, and a concave posterior surface.
